(19) United States
(12) Patent Application Publication
(10) Pub. No.: US 2008/0220463 A1
(43) Pub. Date: Sep. 11, 2008

(54) METHOD OF CELL CHROMATOGRAPHY

(76) Inventors: David J. Beebe, Monona, WI (US); Ivar Meyvantsson, Madison, WI (US)

Correspondence Address: BOYLE FREDRICKSON S.C., 840 North Plankinton Avenue, MILWAUKEE, WI, 53203, US

(21) Appl. No.: 11/683,256

(22) Filed: Mar. 7, 2007

(51) Int. Cl. C12Q 1/02

(52) U.S. Cl. 435/29

(57) ABSTRACT

A method is provided of conducting cell chromatography with a group of cells. Each cell has a fluorescence intensity. The method includes the step of depositing the group of cells into a first chamber for a first predetermined time period such that a first portion of cells of the group of cells attaches to a first surface. The cells unattached to the first surface are removed from the first chamber and deposited into a second chamber for a second predetermined time period. A second portion of cells of the unattached cells attach to a second surface. The cells unattached to the second surface are removed from the second chamber. Thereafter, the fluorescence intensities of the cells attached to the first and second surfaces are compared to a standard.

### REFERENCE TO GOVERNMENT GRANT

This invention was made with United States government support awarded by the following agencies: Army BCRP 144-MQ96 and NIH 144-NB83. The United States has certain rights in this invention.

### FIELD OF THE INVENTION

This invention relates generally to microfluidic devices, and in particular, to a method of cell sorting utilizing a microfluidic platform.

### BACKGROUND AND SUMMARY OF THE INVENTION

For many biological studies, the ability to separate cells is very useful. Typically, cells are separated using a device called a cell sorter. The cell sorter is an automated instrument that separates and rapidly sorts closely related cell populations. By measuring the physical and chemical properties of cells, such as fluorescence, then by physically separating cells while still alive, the cell sorter has become an important tool for biomedical research and clinical medicine.

Two types of cell sorters are most commonly used to isolate cells from complex mixtures, namely, fluorescence activated cell sorters (FACS) and magnetic cell separators. In FACS, live or fixed cells are labeled by fluorescent dyes. Thereafter, individual cells are sorted based upon the specific light scattering and fluorescent characteristics of each cell. Alternatively, magnetic cell separators may be used to sort individual cells. Magnetic separators utilize an affinity group on the surface of a magnetic particle. A suspension of these particles is thoroughly mixed with a preparation of the target molecule or cell. After an incubation period, during which the target binds to an affinity ligand, a powerful magnet is used to immobilize the magnetic particles and their trapped analytes. The unbound material is removed by aspiration and the bound material is washed and detected.

Exquisite specificity notwithstanding, FACS and magnetic bead affinity sorting are not ideal from a biological perspective. As heretofore described, both methods employ antibodies that bind to cell surface antigens. These antigens are often cell signal receptors. As such, the cell perceives a strong signal during separation that may significantly change the cells behavior either transiently or permanently. In addition, FACS execution and magnetic bead elution is often perform in non-physiological buffers that can have detrimental effects on the integrity of the cells. Conventional sorting methods are a confounding factor in cell biological experiments. Hence, a more gentle means of sorting cells would be highly desirable.

Therefore, it is primary object and feature of the present invention to provide a method of cell sorting utilizing a microfluidic platform.

It is a further object and feature of the present invention to provide a method of cell sorting utilizing a microfluidic platform that is simpler and more inexpensive than prior methods.

It is a still further object and feature of the present invention to provide a method of cell sorting utilizing a microfluidic platform that is more gentle on the sorted cells than prior methods.

In accordance with the present invention, a method of conducting cell chromatography is provided. The method includes the step of depositing cells into a first chamber. The first chamber is defined by a first surface. Thereafter, the cells unattached to the first surface are removed from contact with the first surface.

The method may include the additional step of depositing the cells unattached to the first surface into a second chamber. The second chamber is defined by a second surface. Thereafter, the cells unattached to the second surface are removed from the second chamber. The cells unattached to the first surface are removed from the first chamber after a first predetermined time period and the cells unattached to the second surface are removed from the first chamber after a second predetermined time period. The second predetermined time period is greater than the first predetermined time period.

The fluorescence intensity of the cells attached to the first surface versus time is plotted on a graph. In addition, the fluorescence intensity of the cells attached to the second surface versus time is plotted on the graph. The fluorescence intensities of the cells attached to the first and second surfaces are compared to a standard.

In accordance with a further aspect of the present invention, a method of conducting cell chromatography with a group of cells is provided. Each cell has a fluorescence intensity. The group of cells is deposited into a first chamber for a first predetermined time period. A first portion of cells of the group of cells attach to a first surface. The cells unattached to the first surface are removed from the first chamber and deposited into a second chamber for a second predetermined time period. A second portion of cells of the unattached cells attach to a second surface. The cells unattached to the second surface are removed from the second chamber. Thereafter, the fluorescence intensities of the cells attached to the first and second surfaces are compared to a standard.

It is noted that the second predetermined time period is greater than the first predetermined time period and that the first and second channels extend though a microfluidic device. The method may include the additional step of plotting the fluorescence intensities of the cells attached to the first and second surfaces versus time on a graph.

In accordance with a still further aspect of the present invention, a method of conducting cell chromatography with a group of cells is provided. Each cell has a fluorescence intensity. The method includes the step of depositing the group of cells into a first chamber for a first time period. A first portion of cells of the group of cells attach to a first surface. Thereafter, the number of the first portion of cells are compared to a first standard.

The method may include the additional steps of waiting for a second time period such that a second portion of cells of the group of cells attach to the first surface and comparing the second portion of cells to a second standard. Alternatively, the group of cells unattached to the first surface are removed from the first chamber. The group of unattached cells are deposited into a second chamber for a second time period. A second portion of cells of the group of cells attach to a second surface. The number of the second portion of cells are compared to a second standard.

The group of cells unattached to the second surface are removed from the second chamber. It is noted that the second predetermined time period is greater than the first predetermined time period and the first and second channels extend though a microfluidic device. The fluorescence intensities of the first and second portions of cells versus time may be plotted on a graph.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is an isometric view of an exemplary device for effectuating a methodology in accordance with the present invention;

FIG. 2 is a cross-sectional view of the device taken along line 2-2 of FIG. 1;

FIG. 3 is a cross-sectional view of the device taken along line 3-3 of FIG. 2;

FIG. 4 is an isometric view of a cover slip used with the device of FIG. 1; and

FIG. 5 is a graphical representation of a cellular chromatogram indicating cell adhesion properties of different cell types.

### DETAILED DESCRIPTION OF THE DRAWINGS

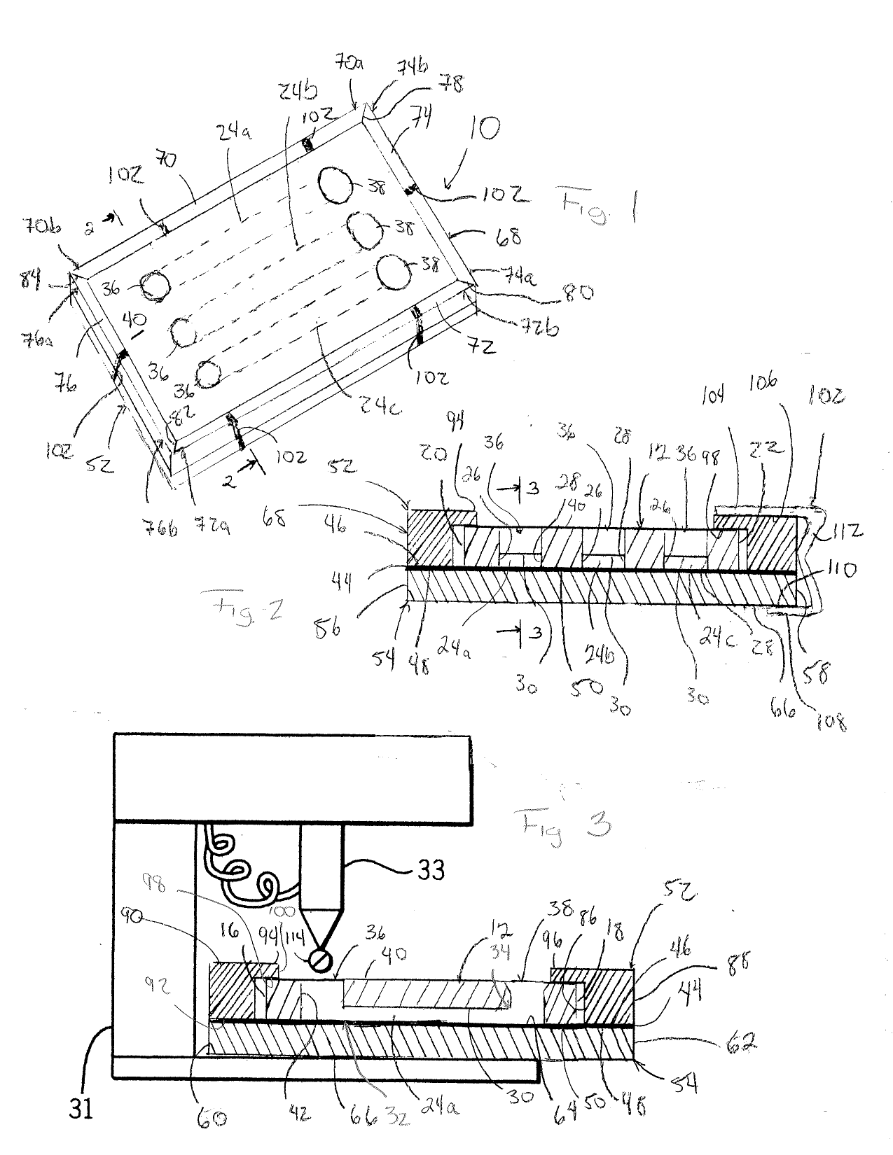

Referring to FIGS. 1-3, an exemplary device for effectuating the methodology of the present invention is generally designated by the reference numeral 10. Device 10 includes cartridge 12. Cartridge 12 defined by first and second ends 16 and 18, respectively, and first and second sides 20 and 22, respectively. A plurality of channels 24a-24c are provided in cartridge 12. It can be appreciated that the number and location of channels in cartridge 12 can vary without deviating from the scope of the present invention. Further, each channel 24a-24c is identical in structure. As such, the following description of channel 24a is understood to describe channels 24b-24c as if fully described herein.

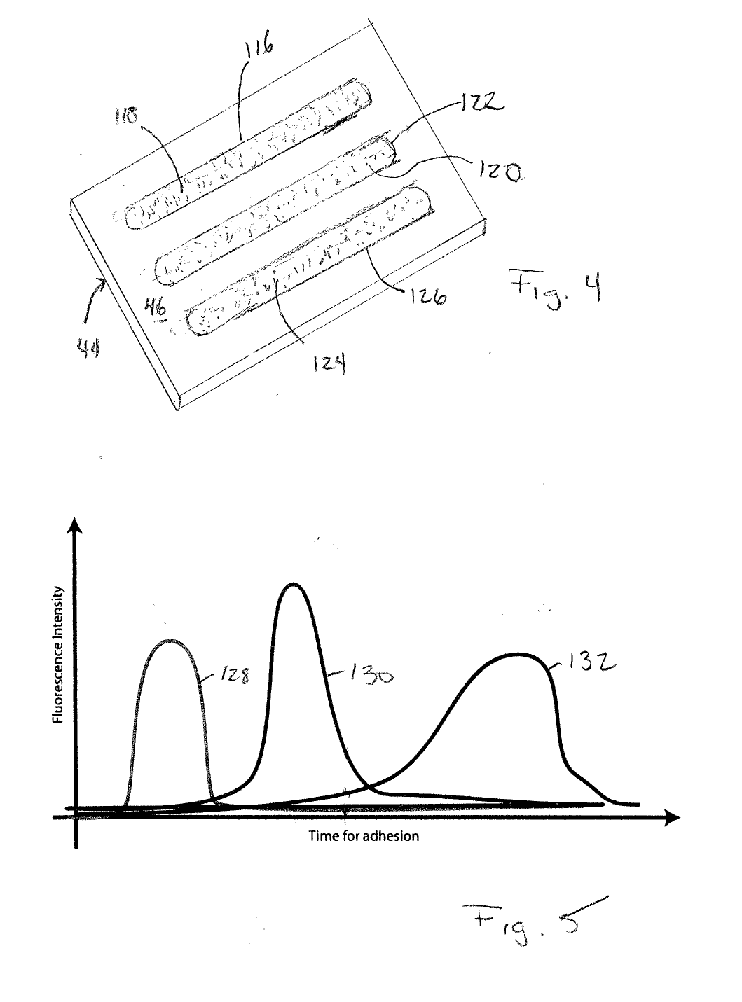

Channel 24a extends along a longitudinal axis and is defined by first and second spaced sidewalls 26 and 28, respectively, and upper wall 30, FIGS. 2-3. Channel 24a further includes first and second ends 32 and 34, respectively, communicate with inlet 36 and outlet 38, respectively. Inlet 36 and outlet 38 communicate with upper surface 40 of cartridge 12. It is contemplated for outlet 38 of channel 24a to have a generally funnel-shaped cross section to allow for robust and easy mating with a micropipette 33 of robotic micropipetting station 31. It is further contemplated for a portion of upper surface 40 of cartridge 12 about inlet 36 or for inner surface 42 defining inlet 36 to be physically or structurally patterned to contain fluid droplets within/adjacent inlet 36 and prevent cross channel contamination with the inlets of channels 24b-24c. Similarly, inlet 36 of channel 24a may have a generally funnel-shaped cross section to allow for robust and easy mating with micropipette 33. In addition, a portion of upper surface 40 of cartridge 12 about outlet 38 or the inner surface defining outlet 38 may be physically or structurally patterned to contain fluid droplets within/adjacent outlet 38 to prevent cross channel contamination.

Device 10 further includes cover slip 44 having upper and lower surfaces 46 and 48, respectively. Upper surface 46 of cover slip 44 is positioned against lower surface 50 of cartridge 12. While it is contemplated to permanently bond upper surface 46 of cover slip 44 to lower surface 50 of cartridge 12, it also contemplated to allow for cover slip 44 to be removed from lower surface 50 of cartridge 12, for reasons hereinafter described. As best seen in FIGS. 2-3, with upper surface 46 of cover slip 44 positioned against lower surface 50 of cartridge 12, upper surface 46 of cover slip 44 partially defines and communicates with channels 24a-24c.

In order to prevent leakage and cross channel contamination between channels 24a-24c along upper surface 46 of cover slip 44, clamping frame 52 is provided. Clamping frame 52 includes substrate 54 defined first and second sides 56 and 58, respectively, and first and second ends 60 and 62, respectively. In the depicted embodiment, substrate 54 has a generally rectangular configuration. However, it can be appreciated that substrate 54 can have other configurations without deviating from the scope of the present invention. Substrate 54 is further defined by upper and lower surfaces 64 and 66, respectively. Upper surface 64 of substrate 54 is adapted for receiving lower surface 48 of cover slip 44 thereon.

Clamping frame 52 further includes frame or bracket 68. In the depicted embodiment, bracket 68 has a generally frame-like configuration and is defined by first and second side elements 70 and 72, respectively, and first and second end elements 74 and 76, respectively. In order to assemble bracket 68, first end 70a of first side element 70 is joined to second end 74b of first end element 74 at corner 78; first end 74a of first end element 74 is joined to second end 72b of second side element 72 at corner 80; first end 72a of second side element 72 is joined to second end 76b of second end element 76 at corner 82; and first end 76a of second end element 76 is joined to second end 70b of first side element 70 at corner 84.

Bracket 68 further includes inner surface 86 and outer surface 88. Inner surface 86 of bracket 68 is defined by the inner surfaces of first and second side elements 70 and 72, respectively, and first and second end elements 74 and 76, respectively. Outer surface 88 of bracket 68 is defined by the outer surfaces of first and second side elements 70 and 72, respectively, and the outer surfaces of first and second end elements 74 and 76, respectively. Inner and outer surfaces 86 and 88, respectively, of bracket 68 are interconnected and spaced by upper and lower surfaces 90 and 92, respectively. Flange 94 projects radially inward from inner surface 86 of bracket 68 and is defined by upper surface 96 that is substantially flush with upper surface 90 of bracket 68 and lower surface 98 that extends from inner surface 86 of bracket 68. Upper and lower surfaces 96 and 98, respectively, of flange 94 are interconnected and spaced by inner edge 100.

In order to assemble device 10, lower surface 48 of cover slip 44 is received on upper surface 64 of substrate 54. Cartridge 12 is positioned on upper surface 46 of cover slip 44. Bracket 68 is deposited on upper surface 64 of substrate 54 such that flange 94 overlaps first and second sides 20 and 22, respectively, of cartridge 12, as well as, first and second ends 16 and 18, respectively. In addition, outer surfaces of first and second side elements 70 and 72, respectively, of bracket 68 are substantially flush with corresponding first and second sides 56 and 58, respectively, of substrate 54 and the outer surfaces of first and second end elements 74 and 76, respectively, of bracket 68 are substantially flush with corresponding first and second ends 60 and 62, respectively, of substrate 54.

It is contemplated to mechanically clamp bracket 68 to substrate 54 so as to prevent leakage and cross channel contamination between channels 24a-24c along upper surface 46 of cover slip 44, but allow removal of bracket 68 from substrate 54. By way of example, screws, rivets, snap connectors or the like may extend through bracket 68 into substrate 54 so as to removably interconnect bracket 68 and substrate 54. Alternatively, a plurality of clamps 102 spaced about the outer periphery of bracket 68 may be used. Each clamp 102 includes upper arm 104 having lower surface 106 engageable with upper surface 90 of bracket 68 and lower arm 108 having an upper surface 110 engageable with lower surface 66 of substrate 54. Biasing leg 112 interconnects upper and lower arms 104 and 108, respectively, and urges upper and lower arms 104 and 108, respectively, toward each other such that clamp 102 retains bracket 68 on substrate 54 and prevents leakage and cross channel contamination between channels 24a-24c along upper surface 46 of cover slip 44.

As is known, the normal adult human body has around 200 types of cells. Each type of cell has different biochemical characteristics related to its function in the body. One category of biochemical characteristics is adhesion. Cells will adhere differently to a surface depending on the molecules it expresses on its surface including integrins and proteoglycans. For example, fibroblasts will adhere more readily to a surface than epithelial cells or adipocytes.

The methodology of the present invention contemplates creating concentrated cell suspension 114 by providing a plurality of unattached floating cells in liquid culture medium. Concentrated cell suspension 114 is loaded into robotic micropipetting station 31 and introduced by micropipette 33 into first channel 24a. Concentrated cell suspension 114 is incubated within first channel 24a for a predetermined time period (e.g. 5 minutes). During the incubation period, a first portion of the cells 118 in concentrated cell suspension 114 adheres to upper surface 46 of cover slip 44 at first location 116, FIG. 4. Using micropipetting station 31, concentrated cells suspension 114 is drawn from first channel 24a and introduced by micropipette 33 into second channel 24b. Concentrated cell suspension 114 is incubated within second channel 24b for a second, predetermined time period (e.g. 10 minutes). During the incubation period, a second portion of the cells 120 in concentrated cell suspension 114 adheres to upper surface 46 of cover slip 44 at second location 122. Using micropipetting station 31, concentrated cells suspension 114 is drawn from second channel 24b and introduced by micropipette 33 into third channel 24c. Concentrated cell suspension 114 is incubated within third channel 24c for a third, predetermined time period (e.g. 15 minutes). During the incubation period, a third portion of the cells 124 in concentrated cell suspension 114 adheres to upper surface 46 of cover slip 44 at third location 126, FIG. 4. It is contemplated to flow the concentrated cell suspension 114 in channels of the device 10 in opposite directions during the incubation periods to, for example, loosen the cells not strongly adhered to upper surface 46 of cover slip 44.

It can be appreciated that the process can be repeated by providing an array of channels in cartridge 12 or by introducing the concentrated cell suspension into additional channels in separate device. The incubation time of the concentrated cell suspension in each channel across the array is increased until such time that all of the cells of interest have attached to upper surface 46 of cover slip 44. Thereafter, in order to determine the number of the cells adhered to cover slip 44, the fluorescence intensities of such cells may be determined. Of course, other mechanisms for measuring the number of cells, such as absorbance or counting, may be used without deviating from the scope of the present invention. Clamps 102 are removed so as to disengage bracket 68 and substrate 54. Bracket 68 and cartridges 12 are removed from cover slip 44 and cover slip 44 is removed from substrate 54 for placement on a fluorometer (not shown). The fluorometer is used to determine the fluorescence intensities of the portions of the cells adhered to upper surface 46 of cover slip 44 at, for example, first, second and third locations 116, 122 and 126, respectively. As a result, each of the portions of cells can be labeled with multiple fluorophore characteristics of relevant cell types (e.g. cluster of differentiation antigens). It can be appreciated that first, second and third locations 116, 122 and 126, respectively, may be treated (such as with a coating) to increase the specificity/adhesion of a certain cell type at the locations. Referring to FIG. 5, the fluorescence intensities associated with specific antigens as a function of incubation time (or chamber number) may be graphically represented. The resulting graph is a cellular chromatogram depicting the cell adhesion properties of different cell types. For example, line 128 may represent blood cell markers, line 130 may represent fibroblast markers and line 132 may represent epithelial cell markers.

By reviewing the graph depicted in FIG. 5, it can be appreciated that a user would be able to differentiate the different cell types that were present in concentrated cell suspension 114. In addition, the graphical illustration shown in FIG. 5 may be compared with standard cell chromatograms to serve as a diagnostic tool for diseases. For example, in epithelial cancers or carcinomas, the cancerous cells change their adhesion properties significantly as the disease progresses, and adhesion properties of cells are directly related to disease stage and prognosis. A comparison of a cancer biopsy to normal tissue from the same patient may provide information on the progression of the carcinoma and help determine the appropriate treatment.

Various modifications to the methodology heretofore described are contemplated as being within the scope of present invention. By way of example, channels 24a-24c may be simultaneously filled with concentrated cell suspension 114. Concentrated cell suspension 114 may be incubated within first channel 24a for the predetermined time period (e.g. 5 minutes), and thereafter, drawn from first channel 24a; concentrated cell suspension 114 may be incubated within second channel 24b for the second, predetermined time period (e.g. 10 minutes), and thereafter, drawn from second channel 24b; and concentrated cell suspension 114 may be incubated within third channel 24c for a third, predetermined time period (e.g. 15 minutes), and thereafter, drawn from third channel 24c. As heretofore described, the fluorescence intensities associated with the adhered cells as a function of incubation time (or chamber number) may be graphically represented and compared to a standard.

Alternatively, first channel 24a may be filled with the concentrated cell suspension. Thereafter, the fluorescence intensities associated with the adhered cells may be measured at predetermined points in time. These measured fluorescence intensities as a function of incubation time may be graphically represented and compared to a standard. In addition, it is contemplated to fill first channel 24a with concentrated cell suspension 114. Concentrated cell suspension 114 is incubated within first channel 24a for a predetermined time period. During the incubation period, a first portion of the cells 118 in concentrated cell suspension 114 adheres to upper surface 46 of cover slip 44 at a first location. Thereafter, the unadhered cells are flowed downstream within first channel 24a and incubated for a second predetermined time period. During the incubation period, a second portion of the cells 118 in concentrated cell suspension 114 adheres to upper surface 46 of cover slip 44 at a second location. The number of cells adhered to the first and second locations of cover slip 44 may be determined.

Various modes of carrying out the invention are contemplated as being within the scope of the following claims particularly pointing out and distinctly claiming the subject matter, which is regarded as the invention.

We claim:

1. A method of conducting cell chromatography, comprising the steps of:
depositing cells into a first chamber, the first chamber being defined by a first surface; and
removing cells unattached to the first surface from contact with the first surface.

2. The method of claim 1 comprising the additional steps of:
depositing the cells unattached to the first surface into a second chamber, the second chamber being defined by a second surface; and
removing cells unattached to the second surface from the second chamber.

3. The method of claim 2 wherein the first surface has a first characteristic and the second surface has a second characteristic.

4. The method of claim 2 wherein the cells unattached to the first surface are removed from the first chamber after a first predetermined time period.

5. The method of claim 4 wherein the cells unattached to the second surface are removed from the first chamber after a second predetermined time period.

6. The method of claim 5 wherein the second predetermined time period is greater than the first predetermined time period.

7. The method of claim 2 wherein the cells attached to the first surface have fluorescence intensities and wherein the method comprises the additional step of plotting the fluorescence intensities of the cells attached to the first surface versus time on a graph.

8. The method of claim 6 wherein the cells attached to the second surface have fluorescence intensities and wherein the method comprises the additional step of plotting the fluorescence intensities of the cells attached to the second surface versus time on the graph.

9. The method of claim 2 wherein the cells attached to the first and second surfaces have fluorescence intensities and wherein the method comprises the additional step of comparing the fluorescence intensities of the cells attached to the first and second surfaces to a standard.

10. The method of claim 1 wherein the cells attached to the first surface have fluorescence intensities and wherein the method comprises the additional step of comparing the fluorescence intensities of the cells attached to the first surface at predetermined time periods to a standard.

11. The method of claim 1 comprising the additional steps of:
bringing the cells unattached to the first surface into contact with a second surface; and
removing cells unattached to the second surface from contact with the second surface.

12. The method of claim 1 wherein the step of depositing the cells in the first chamber includes the additional steps of flowing the cells into the first chamber in a first direction and varying the direction of the flow of cells.

13. A method of conducting cell chromatography with a group of cells, each cell having a fluorescence intensity, comprising the steps of:
depositing the group of cells into a first chamber for a first predetermined time period, a first portion of cells of the group of cells attaching to a first surface;
removing the cells unattached to the first surface from the first chamber;
depositing the cells unattached to the first surface into a second chamber for a second predetermined time period, a second portion of cells of the unattached cells attaching to a second surface;
removing the cells unattached to the second surface from the second chamber; and
comparing the fluorescence intensities of the cells attached to the first and second surfaces to a standard.

14. The method of claim 13 wherein the second predetermined time period is greater than the first predetermined time period.

15. The method of claim 13 comprising the additional steps of plotting the fluorescence intensities of the cells attached to the first and second surfaces versus time on a graph.

16. The method of claim 13 wherein the first and second channels extend though a microfluidic device.

17. A method of conducting cell chromatography with a group of cells, comprising the steps of:
depositing the group of cells into a first chamber for a first time period, a first portion of cells of the group of cells attaching to a first surface; and
comparing the number of the first portion of cells to a first standard.

18. The method of claim 17 comprising the additional steps of:
waiting for a second time period such that a second portion of cells of the group of cells attach to the first surface; and
comparing the number of the second portion of cells to a second standard.

19. The method of claim 17 comprising the additional steps of:
removing the group of cells unattached to the first surface from the first chamber;
depositing the group of unattached cells into a second chamber for a second time period, a second portion of cells of the group of cells attaching to a second surface; and
comparing the number of the second portion of cells to a second standard.

20. The method of claim 19 comprising the additional step of removing the group of cells unattached to the second surface from the second chamber.

21. The method of claim 19 comprising the additional step of providing the first and second chambers in a microfluidic device.

22. The method of claim 19 wherein the second predetermined time period is greater than the first predetermined time period.

23. The method of claim 19 wherein each cell has a fluorescence intensity and wherein the method comprises the additional steps of plotting the fluorescence intensities of the first and second portion of cells versus time on a graph.

24. The method of claim 17 comprising the additional steps of:
bringing the cells unattached to the first surface into contact with a second surface; and
removing cells unattached to the second surface from contact with the second surface.

25. The method of claim 17 wherein the step of depositing the group of cells in the first chamber includes the additional steps of flowing the cells into the first chamber in a first direction and varying the direction of the flow of cells.
